(19) United States
(12) Patent Application Publication
(10) Pub. No.: US 2003/0113869 A1
(43) Pub. Date: Jun. 19, 2003

(54) Human FGF gene and gene expression products

(76) Inventors: Hui Cen, Alameda, CA (US); Pablo D. Garcia, San Francisco, CA (US); Uta Grieshammer, San Francisco, CA (US); Altaf Kassam, Alameda, CA (US); Pauline P. Lee, Contra Costa, CA (US); David Pot, San Francisco, CA (US); Denis Gospodarowicz, Contra Costa, CA (US); Kathleen Martin, Alameda, CA (US)

Correspondence Address: Chiron Corporation, Intellectual Property R338, P.O. Box 8097, Emeryville, CA, 94662-8097, US

(21) Appl. No.: 09/836,960

(22) Filed: Apr. 17, 2001

Related U.S. Application Data
(62) Division of application No. 09/264,851, filed on Mar. 8, 1999.
(60) Provisional application No. 60/077,411, filed on Mar. 9, 1998.
(60) Provisional application No. 60/083,553, filed on Apr. 29, 1998.

(30) Foreign Application Priority Data
Mar. 9, 1999 (US) PCT/US99/05235

(51) Int. Cl. C07H 21/04; C12N 1/18; C12N 1/21; C12N 5/06; C12P 21/02; C07K 14/50

(52) U.S. Cl. 435/69.4; 435/325; 435/252.3; 435/254.1; 536/23.5; 530/399

(57) ABSTRACT

This invention relates to human fibroblast growth factor (FGF 98), and to variants thereof and to polynucleotides encoding FGF 98. The invention also relates to diagnostic and therapeutic agents related to the polynucleotides and proteins, including probes and antibodies.

### REFERENCE TO RELATED APPLICATIONS

This application is a continuation of provisional U.S. Patent Application Serial No. 60/077,411, filed Mar. 9, 1998, and U.S. patent application Ser. No. 60/083,553, filed Apr. 29, 1998.

### TECHNICAL FIELD

The present invention relates to novel nucleic acid sequences encoding a member of the fibroblast growth factor (FGF) family, and to polypeptides encoded by the nucleic acid sequences.

### BACKGROUND OF THE INVENTION

This invention provides a gene of the fibroblast growth factor (FGF) family and protein encoded by the gene. The invention is directed to mRNA expressed by human cells, polynucleotides having coding regions corresponding to the mRNA, protein products of the polynucleotides and polypeptide products of mRNA, and biological function of the polypeptides and proteins.

At least 17 members of the FGF family have been identified, and are discussed in, for example, Ohuchi et al., Development 124:2235-2244 (1994); Ghosh et al., Cell Growth and Differentiation 7:1425-1434 (1996); Gemel et al., Genomics 35:253-257 (1996); Crossley et al., Development 121:439-451 (1995); and Crossley et al., Cell 84:127-136 (1996).

### SUMMARY OF THE INVENTION

The present invention provides a composition comprising an isolated polynucleotide selected from the group consisting of:

(a) a polynucleotide comprising at least eight contiguous nucleotides from nucleotide 550 to 1170 of SEQ ID NO:4;

(b) a polynucleotide that encodes a variant of the polypeptide encoded by (a); and

(c) a polynucleotide encoding a protein expressed by a polynucleotide having the sequence of SEQ ID NO:4.

The invention further provides for the use of the isolated polynucleotides or fragments thereof as diagnostic probes or as primers.

The present invention also provides a composition comprising a polypeptide, wherein said polypeptide is selected from the group consisting of:

(a) a polypeptide comprising at least 6 contiguous amino acids encoded by SEQ ID NO:4 in the region of nucleotide 550 to 1170;

(b) a polypeptide encoded by a polynucleotide comprising SEQ ID NO:4; and

(c) a variant of the protein (a) or (b).

In certain preferred embodiments of the invention, the polynucleotide is operably linked to an expression control sequence. The invention further provides a host cell, including bacterial, yeast, insect and mammalian cells, transformed with the polynucleotide sequence. The invention also provides full-length cDNA and full-length polynucleotides corresponding to SEQ ID NO:4.

Protein and polypeptide compositions of the invention may further comprise a pharmaceutically acceptable carrier. Compositions comprising an antibody that specifically reacts with such protein or polypeptide are also provided by the present invention.

The invention provides methods of using the FGF of the invention for the isolation, regeneration, proliferation, and differentiation of mammalian multipotent neural stem cells, progenitor cells and progeny.

In a preferred embodiment, primary central (CNS) and peripheral nervous system (PNS) cells when treated with FGF 98 of the invention proliferate, have at least a limited self regeneration capacity, and can undergo lineage restriction in response to the local environment.

In a further embodiment, cells produced by treatment with FGF 98 are used to screen drugs such as small molecules, and growth factors, which may affect development, differentiation, survival and or function of CNS and PNS derived neurons and glia.

The invention also provides for the production of large amounts of otherwise minor cell populations of cells to be used for generation of cDNA libraries for the isolation of rare molecules expressed in the precursors cells or progeny; cells produced by treatment may directly express growth factors or other molecules, and conditioned media is screened in assays for novel activities.

The invention further provides for the isolation, self-renewal and survival of mammalian neural stem cells and the differentiation of their progeny.

### BRIEF DESCRIPTION OF THE DRAWINGS

FIG. 1. Western analysis of FGF 98 produced by SF-9 cells infected with different clones of baculovirus expressing FGF 98 (Clone 1 to 6). The highest producers were Clones 2 and 4.

FIG. 2.(A) Elution profile of the CM of SF-9 cells expressing FGF 98 on a Heparin Sepharose (HS) affinity column. FIG. 2(B) shows the OD profile of the 0.5M, 1M and 2M Nad (●-●) fractions together with the bioactivity profile of the various fractions (0) when tested on granulosa cells.

FIG. 3.(A) shows SDS-PAGE analysis of the 2M NaCl fractions followed by Coomassie blue staining, indicating the presence of a single band with a MW of 28 kDA in the peak fractions of the 2M NaCl eluate (frac. 13, 14). FIG. 3(B) shows Western analysis using an anti-peptide FGF 98 antibody of the HS fractions shown in FIG. 2A. A single band with a MW of 28 kD is seen in fractions 12 to 16 corresponding to the 2M NaCl fractions.

FIG. 4. The active HS fractions (Fractions 2-16) were loaded onto a C4 Vydac column (25×0.46 cm, 5 μm particle size, 300 A pore size) equilibrated in 0.1% (v/v) TFA, 20% acetonitrile. Protein was eluted with a 115 min. gradient of 20-100% acetonitrile in 0.1% TFA at a flow rate of 0.6 ml/min. at room temperature. Fractions of 1.2 ml were collected. Aliquots of each fraction were diluted 1 to 10 with 0.2% gelatin in PBS and bioassayed on granulosa cells. A major peak of protein closely associated with a small peak could be observed. Both protein peaks were bioactive.

FIG. 5.(A) SDS-PAGE of the RP-HPLC fractions shown in FIG. 4. When stained by Coomassie blue, both Fractions 22 and 23 gave a single band with a MW of 28 kDA. FIG. 5(B). When analyzed by Western using an FGF 98 anti-peptide antibody both Fractions 22 and 23 were recognized as a single band with a MW of 28 kDa.

FIG. 6. Heparin Sepharose chromatography of a 3 liter batch of medium conditioned by SF-9 cells expressing FGF 98. The CM was adjusted to O.5M NaCl and pH to 7.3. It was applied on a column of Heparin Sepharose equilibrated in 0.5M NaCl 10 mM Tris pH 7.2 on sequentially eluted with 1M and 2M NaCl (A, B).

FIG. 7.(A) SDS-PAGE of the 1M and 2M NaCl fractions of FIG. 6 followed by Coomassie blue staining. FIG. 7(B). Western analysis of the 1M and 2M NaCl fractions of FIG. 6 using a specific FGF 98 antipeptide antibody.

FIG. 8. Mono S ion-exchange chromatography of the HS-purified and active fractions. The bioactive fractions eluted from HS were affinity pooled and diluted 10-fold with 10 mM Tris pH 7.2. Using a 50 ml Super loop, the sample was then applied on a Mono S HR 5/5 column equilibrated in 10 mM Tris pH 7.2-0.2 M NaCl at room temperature. The column was eluted with a multilinear gradient of NaCl (0.2M to 2M NaCl). Absorbency was monitored at 280 nm. Flow rate was 1 ml/min and 1 ml fractions were collected Aliquots of each fraction were diluted 1 to 100 in 0.2% gelatin in PBS, and 10 μl aliquots were bioassayed on granulosa cells in 12 well cluster plates as described.

FIG. 9. Comparison of the biological activity for granulosa cells of FGF 98 (●) and basic FGF(0).

FIG. 10 is a graph showing the effect of FGF 98 on neural stem cell proliferation. Dotted bars represent 6 days in vitro (DIV) and solid bars represent 21 days in vitro (DIV). The treatment consisted of no additions (1), bFGF (2), or FGF 98 of the invention (3).

FIG. 11. Panel A is a photograph of bFGF-treated rat primary hippocampal cell cultures, showing some nestin expression. Panel B shows a separate culture of cells treated with FGF 98 of the invention. More extensive nestin expression is seen.

FIG. 12. Panel A shows FGF 98 mRNA expression in adult brain stem and cerebellum. Panel B shows a high magnification of FGF 98 mRNA expression in motor neurons of the facial nucleus.

### DETAILED DESCRIPTION OF THE INVENTION

Because of their potent activities for promoting growth, proliferation, survival and differentiation of a wide variety of cells and tissue types, FGFs continue to be pursued as therapeutic agents for a number of different indications, including wound healing, such as musculo-skeletal conditions, for example, bone fractures, ligament and tissue repair, tendonitis, bursitis, etc.; skin conditions, for example, burns, cuts, lacerations, bed sores, slow healing ulcers, etc.; tissue protection and repair during myocardial infarction and ischemia, in the treatment of neurological conditions, for example, neuro-degenerative disease and stroke, in the treatment of eye disease, including macular degeneration, and the like.

The fibroblast growth factor (FGF) proteins identified to date belong to a family of signaling molecules that regulate growth and differentiation of a variety of cell types. The significance of FGF proteins to human physiology and pathology relates in part to their key roles in embryogenesis, in blood vessel development and growth, and in bone growth In vitro experiments have demonstrated a role for FGF in regulating cell growth and division of endothelial cells, vascular smooth muscle cells, fibroblasts, and cardiac and skeletal myocytes Other members of the FGF family and their biological roles are described in Crossley et al., Development 121:439-451 (1995); Ohuchi et al., Development 124:2235-2244 (1997); Gemel et al., Genomics 35:253-257 (1996); and Ghosh et al., Cell Growth and Differentiation 7:1425-1434 (1996).

FGF proteins are also significant to human health and disease because of a role in cancer cell growth. For example, FGF-8 was identified as an androgen-induced growth factor in breast and prostate cancer cells. (Tanaka et al., FEBS Lett. 363:226-230 (1995) and P.N.A.S. 89:8928-8932 (1992)).

A member of the FGF family is described here, wherein the FGF protein is expressed at high levels in adult mouse brain, and plays a protective role in neuronal tissue. A polynucleotide encoding the FGF of the invention has the sequence as shown in SEQ ID NO:1. The polynucleotide was identified as encoding a member of the FGF family by the conserved regions throughout the amino acid sequence and by the regions of homology shared by the polynucleotide and genes encoding known FGF proteins.

The FGF of the invention shares greatest homology with FGF-8, which plays a variety of roles, including outgrowth and patterning in mouse embryo development (Crossley et al., Development 121:439-451 (1995), and midbrain development (Crossley et al., Nature 380:66-68 (1996). Eight FGF-8 isoforms have been identified in mice, generated by alternative splicing, and may be functionally redundant. FGF-8b is 100% identical in mice and humans. Blunt et al., J. Biol. Chem. 272:3733-3738 (1997). FGF-8 and FGF-10 function in a coordinate manner during chick limb development. Ohuchi et al., Development 124:2235-2244 (1997).

As the above brief discussion illustrates, the knowledge about the roles played by various FGF proteins continues to grow, but is by far incomplete.

The present invention adds to this knowledge by disclosing that the FGF of SEQ ID NO:5 plays a role in adult mouse brain and has a protective role in neuronal tissue. The FGF 98 increased the number of neuronal cells at six and twenty days in vitro. Furthermore, unlike bFGF-treated cells, few FGF 98-treated cells die under the conditions used herein. FGF 98 can be used for ex vivo expansion of precursor cells in the nervous system. Transplant or engraftment of the cells can be used as a therapy for diseases in which specific populations of neurons are lost due to degeneration such as, for example, Parkinson's disease and Alzheimer's disease.

The invention therefore is based upon the identification, isolation and sequencing of a new fibroblast growth factor (FGF 98). This growth factor has been found to promote cell survival and, in particular, the survival of neurons.

Reference to FGF 98 herein is intended to be construed to include growth factors of any origin which are substantially homologous to and which are biologically equivalent to the FGF 98 characterized and described herein Such substantially homologous growth factors may be native to any tissue or species and, similarly, biological activity can be characterized in any of a number of biological assay systems.

The terms “biologically equivalent” are intended to mean that the compositions of the present invention are capable of demonstrating some or all of the same growth properties in a similar fashion, not necessarily to the same degree as the FGF 98 isolated as described herein or recombinantly produced human FGF 98 of the invention.

By “substantially homologous” it is meant that the degree of homology of human FGF 98 to FGF 98 from any species is greater than that between FGF 98 and any previously reported member of the FGF family.

Sequence identity or percent identity is intended to mean the percentage of same residues between two sequences, referenced to human FGF when determining percent identity with non-human FGF 98, referenced to FGF 98 when determining percent identity with non-FGF 98 growth factors, when the two sequences are aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 (Dayhoff et al, in Atlas of Protein Sequence and Structure, Dayhoff, Ed., NDRF, Washington, Vol. 5, suppl. 3, p 345, 1978).

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human FGF 98 when determining percent conservation with non-human FGF 98, referenced to FGF 98 when determining percent conservation with non-FGF 98 growth factors. Conservative amino acid changes satisfying this requirement are: R-K; E-D, Y-F, L-M; V-I, Q-H.

FGF 98 can also include hybrid and modified forms of FGF 98 including fusion proteins and FGF 98 fragments and hybrid and modified forms in which certain amino acids have been deleted or replaced and modifications such as where one or more amino acids have been changed to a modified amino acid or unusual amino acid and modifications such as glycosylations so long as the hybrid or modified form retains the biological activity of FGF 98. By retaining the biological activity, it is meant that neuronal survival is promoted, although not necessarily at the same level of potency as that of the FGF 98 isolated as described herein or that of the recombinantly produced human FGF 98.

Also included within the meaning of substantially homologous is any FGF 98 which may be isolated by virtue of cross-reactivity with antibodies to the FGF 98 described herein or whose encoding nucleotide sequences including genomic DNA, mRNA or cDNA may be isolated through hybridization with the complementary sequence of genomic or subgenomic nucleotide sequences or cDNA of the FGF 98 herein or fragments thereof. It will also be appreciated by one skilled in the art that degenerate DNA sequences can encode human FGF 98 and these are also intended to be included within the present invention as are allelic variants of FGF 98.

Growth factors are thought to act at specific receptors. According to the invention, FGF 98 and as yet unknown members of this family of growth factors act through specific receptors having distinct distributions as has been shown for other growth factor families.

A preferred FGF 98 of the present invention has been identified and isolated in purified form as described. Also preferred is FGF 98 prepared by recombinant DNA technology. By “pure form” or “purified form” or “substantially purified form” it is meant that an FGF 98 composition is substantially free of other proteins which are not FGF 98.

Recombinant human FGF 98 may be made by expressing the DNA sequences encoding FGF 98 in a suitable transformed host cell. Using methods well known in the art, the DNA encoding FGF 98 may be linked to an expression vector, transformed into a host cell and conditions established that are suitable for expression of FGF 98 by the transformed cell.

Any suitable expression vector may be employed to produce recombinant human FGF 98 such as expression vectors for use in insect cells. Baculovirus expression systems can also be employed. A preferable method is expression in insect cells, such as Tr5 or Sf9 cells, using baculovirus vector, as described in Example 1.

The present invention includes nucleic acid sequences including sequences that encode human FGF 98. Also included within the scope of this invention are sequences that are substantially the same as the nucleic acid sequences encoding FGF 98. Such substantially the same sequences may, for example, be substituted with codons more readily expressed in a given host cell such as E. coli according to well known and standard procedures. Such modified nucleic acid sequences are included within the scope of this invention.

Specific nucleic acid sequences can be modified by those skilled in the art and, thus, all nucleic acid sequences that code for the amino acid sequences of FGF 98 can likewise be so modified. The present invention thus also includes nucleic acid sequence which will hybridize with all such nucleic acid sequences—or complements of the nucleic acid sequences where appropriate—and encode a polypeptide having the cell survival promoting activities disclosed herein. The present invention also includes nucleic acid sequences that encode for polypeptides that have neuronal survival promoting activity and that are recognized by antibodies that bind to FGF 98. A preferred method for raising antibody is described in Example 9.

The present invention also encompasses vectors comprising expression regulatory elements operably linked to any of the nucleic acid sequences included within the scope of the invention. This invention also includes host cells—of any variety—that have been transformed with vectors comprising expression regulatory elements operably linked to any of the nucleic acid sequences included within the scope of the present invention.

Methods are also provided herein for producing FGF 98. Preparation can be by isolation from conditioned medium from a variety of cell types so long as the cell type produces FGF 98. A second and preferred method involves utilization of recombinant methods by isolating or obtaining a nucleic acid sequence encoding FGF 98, cloning the sequence along with appropriate regulatory sequences into suitable vectors and cell types, and expressing the sequence to produce FGF 98.

Although FGF 98 has been described on the basis of its ability to promote the survival of particular cells, this factor will act on other neuronal cell types as well. For example, FGF 98 is shown herein to enhance nestin expression. Thus, it is likely that FGF 98 can act on a wide variety of central and peripheral neuronal cell types.

It is also likely that FGF 98 will act on non-neuronal cells to promote their survival, growth or function. This expectation is based upon the activity of known growth factors. Members of the FGF family act on many cell types of different function and embryologic origin.

The inventors herein have identified several non-neuronal tissues in which FGF 98 is expressed including lung, heart, developing bone, and chondrocytes. This suggests a role for FGF 98 in, for example, maintenance of musculoskeletal function and treatment of related disease; correction of congenital defects; and treatment cardiomyopathies.

The present invention also includes therapeutic or pharmaceutical compositions comprising FGF 98 in an effective amount for treating patients with cellular degeneration and a method comprising administering a therapeutically effective amount of FGF 98. These compositions and methods are useful for treating a number of degenerative diseases. Where the cellular degeneration involves neuronal degeneration, the diseases include, but are not limited to peripheral neuropathy, amyotrophic lateral sclerosis, Alzheimer's disease, Parkinson's disease, Huntington's disease, ischemic stroke, acute brain injury, acute spinal chord injury, nervous system tumors, multiple sclerosis, peripheral nerve trauma or injury, exposure to neurotoxins, metabolic diseases such as diabetes or renal dysfunctions and damage caused by infectious agents. Where the cellular degeneration involves bone marrow cell degeneration, the diseases include, but are not limited to disorders of insufficient blood cells such as, for example, leukopenias including eosinopenia and/or basopenia, lymphopenia, monocytopenia, neutropenia, anemias, thrombocytopenia as well as an insufficiency of stem cells for any of the above. The above cells and tissues can also be treated for depressed function.

The compositions and methods herein can also be useful to prevent degeneration and/or promote survival in other non-neuronal tissues as well. One skilled in the art can readily determine using a variety of assays known in the art for identifying whether FGF 98 would be useful in promoting survival or functioning in a particular cell type, such as cardiac cells.

In certain circumstances, it may be desirable to modulate or decrease the amount of FGF 98 expressed. Thus, in another aspect of the present invention, FGF 98 anti-sense oligonucleotides can be made and a method utilized for diminishing the level of expression of FGF 98 by a cell comprising administering one or more FGF 98 anti-sense oligonucleotides. By FGF 98 anti-sense oligonucleotides reference is made to oligonucleotides that have a nucleotide sequence that interacts through base pairing with a specific complementary nucleic acid sequence involved in the expression of FGF 98 such that the expression of FGF 98 is reduced. Preferably, the specific nucleic acid sequence involved in the expression of FGF 98 is a genomic DNA molecule or mRNA molecule that encodes FGF 98. This genomic DNA molecule can comprise regulatory regions of the FGF 98 gene, or the coding sequence for mature FGF 98 protein. The term complementary to a nucleotide sequence in the context of FGF 98 antisense oligonucleotides and methods therefor means sufficiently complementary to such a sequence as to allow hybridization to that sequence in a cell, i.e., under physiological conditions. The FGF 98 antisense oligonucleotides preferably comprise a sequence containing from about 8 to about 100 nucleotides and more preferably the FGF 98 antisense oligonucleotides comprise from about 15 to about 30 nucleotides. The FGF 98 antisense oligonucleotides can also contain a variety of modifications that confer resistance to nucleolytic degradation such as, for example, modified internucleoside linages (Uhlmann and Peyman, Chemical Reviews 90:543-548 1990; Schneider and Banner, Tetrahedron Lett. 31:335, 1990 which are incorporated by reference), modified nucleic acid bases and/or sugars and the like.

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation. For treating tissues in the central nervous system, administration can be by injection or infusion into the cerebrospinal fluid (CSF). When it is intended that FGF 98 be administered to cells in the central nervous system, administration can be with one or more agents capable of promoting penetration of FGF 98 across the blood-brain barrier.

FGF 98 can also be linked or conjugated with agents that provide desirable pharmaceutical or pharmacodynamic properties. For example, FGF 98 can be coupled to any substance known in the art to promote penetration or transport across the blood-brain barrier such as an antibody to the transferrin receptor, and administered by intravenous injection (See, for example, Friden et al., Science 259:373-377, 1993 which is incorporated by reference). Furthermore, FGF 98 can be stably linked to a polymer such as polyethylene glycol to obtain desirable properties of solubility, stability, half-life and other pharmaceutically advantageous properties. (See, for example, Davis et al., Enzyme Eng. 4:169-73, 1978; Burnham, Am. J. Hosp. Pharm. 51:210-218, 1994 which are incorporated by reference).

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. FGF 98 can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing FGF 98 are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, FGF 98 may be therapeutically administered by implanting into patients vectors or cells capable of producing a biologically-active form of FGF 98 or a precursor of FGF 98, i.e., a molecule that can be readily converted to a biological-active form of FGF 98 by the body. In one approach cells that secrete FGF 98 may be encapsulated into semipermeable membranes for implantation into a patient. The cells can be cells that normally express FGF 98 or a precursor thereof or the cells can be transformed to express FGF 98 or a precursor thereof. It is preferred that the cell be of human origin and that the FGF 98 be human FGF 98 when the patient is human. However, the formulations and methods herein can be used for veterinary as well as human applications and the term “patient” as used herein is intended to include human and veterinary patients.

Cells can be grown ex vivo for use in transplantation or engraftment into patients (Muench et al., Leuk. & Lymph. 16:1-11, 1994 which is incorporated by reference). In another embodiment of the present invention, FGF 98 is used to promote the ex vivo expansion of a cells for transplantation or engraftment. Current methods have used bioreactor culture systems containing factors such as erythropoietin, colony stimulating factors, stem cell factor, and interleukins to expand hematopoietic progenitor cells for erythrocytes, monocytes, neutrophils, and lymphocytes (Verfaillie, Stem Cells 12:466-476, 1994 which is incorporated by reference). These stem cells can be isolated from the marrow of human donors, from human peripheral blood, or from umbilical cord blood cells. The expanded blood cells are used to treat patients who lack these cells as a result of specific disease conditions or as a result of high dose chemotherapy for treatment of malignancy (George, Stem Cells 12(Suppl 1):249-255, 1994 which is incorporated by reference). In the case of cell transplant after chemotherapy, autologous transplants can be performed by removing bone marrow cells before chemotherapy, expanding the cells ex vivo using methods that also function to purge malignant cells, and transplanting the expanded cells back into the patient following chemotherapy (for review, see Rummel and Van Zant, J. Hematotherapy 3:213-218, 1994 which is incorporated by reference). Since FGF 98 is expressed in the developing animal in blood, bone marrow and liver, tissues where proliferation and differentiation of progenitor cells occur, it is believed that FGF 98 can function to regulate the proliferation of hematopoietic stem cells and the differentiation of mature hematopoietic cells. Thus, the addition of FGF 98 to culture systems used for ex vivo expansion of cells could stimulate the rate at which certain populations of cells multiply or differentiate, and improve the effectiveness of these expansion systems in generating cells needed for transplant.

It is also believed that FGF 98 can be used for the ex vivo expansion of precursor cells in the nervous system. Transplant or engraftment of cells is currently being explored as a therapy for diseases in which certain populations of neurons are lost due to degeneration such as, for example, in Parkinson's disease (Bjorklund, Curr. Opin. Neurobiol. 2:683-689, 1992 which is incorporated by reference). Neuronal precursor cells can be obtained from animal or human donors or from human fetal tissue and then expanded in culture using FGF 98 or other growth factors. These cells can then be engrafted into patients where they would function to replace some of the cells lost due to degeneration. Because neurotrophins have been shown to be capable of stimulating the survival and proliferation of neuronal precursors cells such as, for example, NT-3 stimulation of sympathetic neuroblast cells (Birren et al., Develop. 119:597-610, 1993 which is incorporated by reference), FGF 98 could also function in similar ways during the development of the nervous system and could be useful in the ex vivo expansion of neuronal cells.

In a number of circumstances it would be desirable to determine the levels of FGF 98 in a patient. The identification of FGF 98 along with the present report showing that FGF 98 is expressed by a number of tissues provides the basis for the conclusion that the presence of FGF 98 serves a normal physiologic function related to cell growth and survival. Indeed, other neurotrophic factors are known to play a role in the function of neuronal and non-neuronal tissues. (For review see Scully and Otten, Cell Bol. Int. 19:459-469, 1995; Otten and Gadient, Int. J. Devl. Neurosciences 13:147-151, 1995 which are incorporated by reference). Endogenously produced FGF 98 may also play a role in certain disease conditions, particularly where there is cellular degeneration such as in neurodegenerative conditions or diseases. Other neurotrophic factors are known to change during disease conditions. For example, in multiple sclerosis, levels of NGF protein in the cerebrospinal fluid are increased during acute phases of the disease (Bracci-Laudiero et al., Neuroscience Lett. 147:9-12, 1992 which is incorporated by reference) and in systemic lupus erythematosus there is a correlation between inflammatory episodes and NGF levels in sera (Bracci-Laudiero et al. NeuroReport 4:563-565, 1993 which is incorporated by reference).

Given that FGF 98 is expressed in developing bone and in heart tissue, it is likely that the level of FGF 98 may be altered in a variety of conditions and that quantification of FGF 98 levels would provide clinically useful information. Furthermore, in the treatment of degenerative conditions, compositions containing FGF 98 can be administered and it would likely be desirable to achieve certain target levels of FGF 98 in sera, in cerebrospinal fluid or in any desired tissue compartment. It would, therefore, be advantageous to be able to monitor the levels of FGF 98 in a patient. Accordingly, the present invention also provides methods for detecting the presence of FGF 98 in a sample from a patient.

The term “detection” as used herein in the context of detecting the presence of FGF 98 in a patient is intended to include the determining of the amount of FGF 98 or the ability to express an amount of FGF 98 in a patient, the distinguishing of FGF 98 from other growth factors, the estimation of prognosis in terms of probable outcome of a degenerative disease and prospect for recovery, the monitoring of the FGF 98 levels over a period of time as a measure of status of the condition, and the monitoring of FGF 98 levels for determining a preferred therapeutic regimen for the patient.

To detect the presence of FGF 98 in a patient, a sample is obtained from the patient. The sample can be a tissue biopsy sample or a sample of blood, plasma, serum, CSF or the like. FGF 98 is expressed in a variety of tissues as discussed in Example 8. Samples for detecting FGF 98 can be taken from any of these tissues. When assessing peripheral levels of FGF 98, it is preferred that the sample be a sample of blood, plasma or serum. When assessing the levels of FGF 98 in the central nervous system a preferred sample is a sample obtained from cerebrospinal fluid.

In some instances it is desirable to determine whether the FGF 98 gene is intact in the patient or in a tissue or cell line within the patient. By an intact FGF 98 gene it is meant that there are no alterations in the gene such as point mutations, deletions, insertions, chromosomal breakage, chromosomal rearrangements and the like wherein such alteration might alter production of FGF 98 or alter its biological activity, stability or the like to lead to disease processes or susceptibility to cellular degenerative conditions. Thus, in one embodiment of the present invention a method is provided for detecting and characterizing any alterations in the FGF 98 gene. The method comprises providing an oligonucleotide that contains the FGF 98 cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize to the FGF 98 gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, TaqI and AluI. Using the Southern blot protocol, which is well known in the art, this assay determines whether a patient or a particular tissue in a patient has an intact FGF 98 gene or an FGF 98 gene abnormality.

Hybridization to an FGF 98 gene would involve denaturing the chromosomal DNA to obtain a single-stranded DNA; contacting the single-stranded DNA with a gene probe associated with the FGF 98 gene sequence; and identifying the hybridized DNA-probe to detect chromosomal DNA containing at least a portion of a human FGF 98 gene.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

The FGF 98 gene probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

FGF 98 gene abnormalities can also be detected by using the PCR method and primers that flank or lie within the FGF 98 gene. The PCR method is well known in the art. Briefly, this method is performed using two oligonucleotide primers which are capable of hybridizing to the nucleic acid sequences flanking a target sequence that lies within an FGF 98 gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, the method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising FGF 98 or pre-pro FGF 98 or a fragment thereof is then directly sequenced and analyzed by comparison of the sequence with the sequences disclosed herein to identify alterations which might change activity or expression levels or the like.

In another embodiment, a method for detecting FGF 98 is provided based upon an analysis of tissue expressing the FGF 98 gene. Certain tissues such as those identified below in Example 8 have been found to express the FGF 98 gene. The method comprises hybridizing a polynucleotide to mRNA from a sample of tissues that normally express the FGF 98 gene. The sample is obtained from a patient suspected of having an abnormality in the FGF 98 gene or in the FGF 98 gene of particular cells. The polynucleotide comprises SEQ ID NO:4 or a derivative thereof or a fragment thereof.

To detect the presence of mRNA encoding FGF 98 protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding FGF 98 protein or a derivative of the cDNA as a probe, high stringency conditions can be used in order to prevent false positives, that is the hybridization and apparent detection of FGF 98 nucleotide sequences when in fact an intact and functioning FGF 98 gene is not present. When using sequences derived from the FGF 98 cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. (Sambrook, et al., 1989, supra).

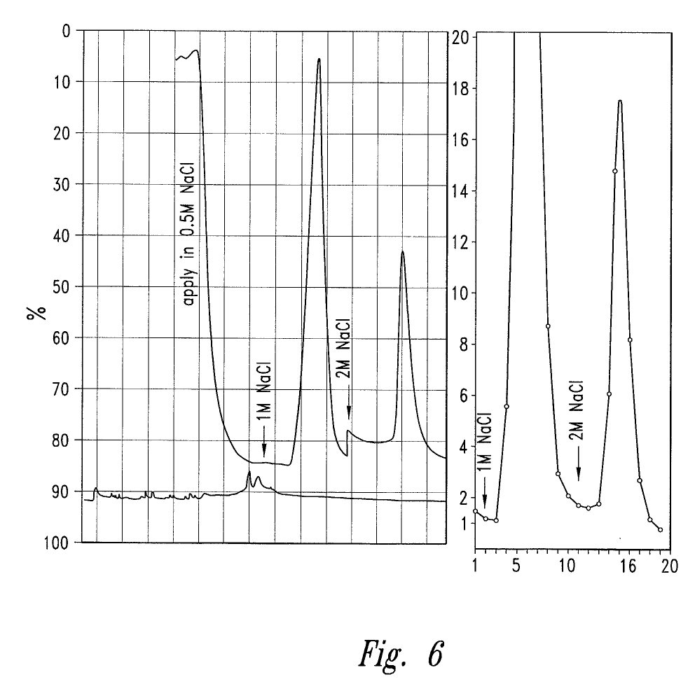

In order to increase the sensitivity of the detection in a sample of mRNA encoding the FGF 98 protein, the technique of reverse transcription/polymerization chain reaction (RT/PCR) can be used to amplify cDNA transcribed from mRNA encoding the FGF 98 protein. The method of RT/PCR is well known in the art (see example 9 and FIG. 6 below).

The RT/PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and FGF 98 specific primers. (Belyavsky et al., Nucl. Acid Res. 17:2919-2932, 1989; Krug and Berger, Methods in Enzymology, 152:316-325, Academic Press, NY, 1987 which are incorporated by reference).

The polymerase chain reaction method is performed as described above using two oligonucleotide primers that are substantially complementary to the two flanking regions of the DNA segment to be amplified.

Following amplification, the PCR product is then electrophoresed and detected by ethidium bromide staining or by phosphoimaging.

The present invention further provides for methods to detect the presence of the FGF 98 protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. (for example, see Basic and Clinical Immunology, 217-262, Sites and Terr, eds., Appleton & Lange, Norwalk, Conn., 1991 which is incorporated by reference). Preferred are binder-ligand immunoassay methods including reacting antibodies with an epitope or epitopes of the FGF 98 protein and competitively displacing a labeled FGF 98 protein or derivative thereof. A preferred antibody is prepared according to Example 9.

As used herein, a derivative of the FGF 98 protein is intended to include a polypeptide in which certain amino acids have been deleted or replaced or changed to modified or unusual amino acids wherein the FGF 98 derivative is biologically equivalent to FGF 98 and wherein the polypeptide derivative cross-reacts with antibodies raised against the FGF 98 protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Polyclonal or monoclonal antibodies to the FGF 98 protein or an epitope thereof can be made for use in immunoassays by any of a number of methods known in the art. By epitope reference is made to an antigenic determinant of a polypeptide. An epitope could comprise 3 amino acids in a spacial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, usually a rabbit or a mouse. (See Example 9).

Oligopeptides can be selected as candidates for the production of an antibody to the FGF 98 protein based upon the oligopeptides lying in hydrophilic regions, which are thus likely to be exposed in the mature protein. A preferred oligopeptide is KRYPKGQPELQKPFK (SEQ ID NO:6).

Antibodies to FGF 98 can also be raised against oligopeptides that include one or more of the conserved regions identified herein such that the antibody can cross-react with other family members. Such antibodies can be used to identify and isolate the other family members.

Methods for preparation of the FGF 98 protein or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system (E. I. du Pont de Nemours Company, Wilmington, Del.) (Caprino and Han, J. Org Chem 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified FGF 98 protein usually by ELISA or by bioassay based upon the ability to block the action of FGF 98 on neurons or other cells. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating an over expression of the protein. Thus, another aspect of the present invention provides for a method for preventing or treating diseases involving over expression of the FGF 98 protein by treatment of a patient with specific antibodies to the FGF 98 protein.

Specific antibodies, either polyclonal or monoclonal, to the FGF 98 protein can be produced by any suitable method known in the art as discussed above. For example, murine or human monoclonal antibodies can be produced by hybridoma technology or, alternatively, the FGF 98 protein, or an immunologically active fragment thereof, or an anti-idiotypic antibody, or fragment thereof can be administered to an animal to elicit the production of antibodies capable of recognizing and binding to the FGF 98 protein. Such antibodies can be from any class of antibodies including, but not limited to IgG, IgA, IgM, IgD, and IgE or in the case of avian species, IgY and from any subclass of antibodies.

Polypeptides encoded by the instant polynucleotides and corresponding full-length genes can be used to screen peptide libraries, protein libraries, small molecule libraries, and phage display libraries, and other known methods, to identify analogs or antagonists.

Native FGF polypeptides may play a role in cancer. For example, FGF family members can induce marked morphological transformation of NIH 3T3 cells, and exhibit strong tumorigenicity in nude mice. Angiogenic activity has been exhibited by FGF family members. Thus, inhibitors of FGF can be used to treat cancer, such as prostate.

A library of peptides may be synthesized following the methods disclosed in U.S. Pat. No. 5,010,175, and in PCT No. WO 91/17823. As described below in brief, a mixture of peptides is prepared, which is then screened to identify the peptides exhibiting the desired signal transduction and receptor binding activity. According to the method of the '175 patent, a suitable peptide synthesis support (e.g., a resin) is coupled to a mixture of appropriately protected, activated amino acids. The concentration of each amino acid in the reaction mixture is balanced or adjusted in inverse proportion to its coupling reaction rate so that the product is an equimolar mixture of amino acids coupled to the starting resin. The bound amino acids are then deprotected, and reacted with another balanced amino acid mixture to form an equimolar mixture of all possible dipeptides. This process is repeated until a mixture of peptides of the desired length (e.g., hexamers) is formed. Note that one need not include all amino acids in each step: one may include only one or two amino acids in some steps (e.g., where it is known that a particular amino acid is essential in a given position), thus reducing the complexity of the mixture. After the synthesis of the peptide library is completed, the mixture of peptides is screened for binding to the selected polypeptide. The peptides are then tested for their ability to inhibit or enhance activity. Peptides exhibiting the desired activity are then isolated and sequenced.

The method described in PCT No. WO 91/17823 is similar. However, instead of reacting the synthesis resin with a mixture of activated amino acids, the resin is divided into twenty equal portions (or into a number of portions corresponding to the number of different amino acids to be added in that step), and each amino acid is coupled individually to its portion of resin. The resin portions are then combined, mixed, and again divided into a number of equal portions for reaction with the second amino acid. In this manner, each reaction may be easily driven to completion. Additionally, one may maintain separate “subpools” by treating portions in parallel, rather than combining all resins at each step. This simplifies the process of determining which peptides are responsible for any observed receptor binding or signal transduction activity.

In such cases, the subpools containing, e.g., 1-2,000 candidates each are exposed to one or more polypeptides of the invention. Each subpool that produces a positive result is then resynthesized as a group of smaller subpools (sub-subpools) containing, e.g., 20-100 candidates, and reassayed. Positive sub-subpools may be resynthesized as individual compounds, and assayed finally to determine the peptides that exhibit a high binding constant. These peptides can be tested for their ability to inhibit or enhance the native activity. The methods described in PCT No. WO 91/7823 and U.S. Pat. No. 5,194,392 (herein incorporated by reference) enable the preparation of such pools and subpools by automated techniques in parallel, such that all synthesis and resynthesis may be performed in a matter of days.

Peptide agonists or antagonists are screened using any available method, such as signal transduction, antibody binding, receptor binding and mitogenic assays. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide may require concentrations equal to or greater than the native concentration, while inhibitors capable of binding irreversibly to the polypeptide may be added in concentrations on the order of the native concentration.

The end results of such screening and experimentation will be at least one novel polypeptide binding partner, such as a receptor, encoded by a gene or a cDNA of the invention, and at least one peptide agonist or antagonist of the novel binding partner. Such agonists and antagonists can be used to modulate, enhance, or inhibit receptor function in cells to which the receptor is native, or in cells that possess the receptor as a result of genetic engineering. Further, if the novel receptor shares biologically important characteristics with a known receptor, information about agonist/antagonist binding may help in developing improved agonists/antagonists of the known receptor.

The therapeutic FGF 98 polynucleotides and polypeptides of the present invention may be utilized in gene delivery vehicles. The gene delivery vehicle may be of viral or non-viral origin (see generally, Jolly, Cancer Gene Therapy 1:51-64 (1994), Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250, ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et al., P.N.A.S. 90:10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. :215-219 (1994); Kass-Eisler et al., P.N.A.S. 90:11498-11502 (1993); Guzman et al., Circulation 88:2838-2848 (1993); Guzman et al, Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al, Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992) may be employed.

Other gene delivery vehicles and methods may be employed, including polycationic condensed DNA linked or unlinked to killed adenovirus alone, for example Curiel, Hum. Gene Ther. 3:147-154 (1992); ligand-linked DNA, for example see Wu, J. Biol. Chem. 264:16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91(24): 11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No 5,149,655; use of ionizing radiation for activating transferred gene, as described in U.S. Pat. No. 5,206,152 and PCT Patent Publication No. WO 92/11033.

FGF has been implicated in diseases characterized by loss of function, inadequate function/number, abnormal function or death of cells, tissues or organs for which function or survival can be prolonged/rescued, and abnormalities reversed or prevented by therapy with FGF.

According to the invention, FGF 98 is likely to play a role in degenerative and other neurological diseases (disease characterized by loss of function or death of CNS cells, including neurons, glial cells, astrocytes, and others), in which therapy may prolong survival of cells or rescue/perserve function. Examples include Parkinson's disease, Alzheimers or other dementia, stroke, trauma, sequelae of infection, epilepsy, cranian nerve injury or dysfunction. FGF 98 may also be useful in treatment of eye disease characterized by similar responses of retinal cells, structural cells, nerve cells, etc., to administration of the molecule. Examples include Retinitis Pigmentosa, age-related macular degeneration, retinal detachment, ischemic retinal disease, etc.

FGF 98 may be useful in treatment of disorders of hearing, smell or taste due to loss/dysfunction of cells responsive to the molecule, including cranial or peripheral nerves, sensory cells, supporting cells, etc., and in treatment of peripheral motor or sensor nerve disease characterized by loss of function or death of neurons, nerves, other CNS or PNS cells or supporting cells: Amyotrophic Lateral Sclerosis, motor or Sensory Neuropathies (due to many causes; viral infections, metabolic disease-diabetes, autoimmune insult, idiopathic, degenerative, etc.) trauma.

Ischemic vascular disease may be amenable to FGF 98 treatment, wherein the disease is characterized by inadequate blood flow to an organ(s). Treatment may induce therapeutic angiogenesis or preserve function/survival of cells (myocardial ischemia/infarction, peripheral vascular disease, renal artery disease, stroke). Cardiomyopathies characterized by loss of function or death of cardiac myocytes or supporting cells in the heart (congestive heart failure, myocarditis) may also be treated using FGF 98, as can musculoskeletal disease characterized by loss of function, inadequate function or death of skeletal muscle cells, bone cells or supporting cells. Examples include skeletal myopathies, bone disease, and arthritis.

FGF 98 polynucleotides and polypeptides may aid in correction of congenital defects due to loss of FGF 98 molecule or its function (heart, lung, brain, limbs, kidney, etc.).

Treatment of wound healing is yet another use of FGF 98 polypeptides and polynucleotides, either due to trauma, disease, medical or surgical treatment, including regeneration of cell populations and tissues depleted by these processes. Examples include liver regeneration, operative wound healing, re-endothelialization of injured blood vessels, healing of traumatic wounds, healing of ulcers due to vascular, metabolic disease, etc., bone fractures, loss of cells due to inflammatory disease, etc.

FGF 98 may also be used in screens to identify drugs for treatment of cancers which involve over activity of the molecule, or new targets which would be useful in the identification of new drugs.

For all of the preceding embodiments, the clinician will determine, based on the specific condition, whether FGF 98 polypeptides or polynucleotides, antibodies to FGF 98, or small molecules such as peptide analogues or antagonists, will be the most suitable form of treatment. These forms are all within the scope of the invention.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLES

### Example 1

### Expression of FGF 98 in Insect Cells

The baculovirus vectors for expression of FGF 98 were generated by PCR of the coding region from a Bluescript vector containing the cDNA for FGF 98, prepared from a human heart cDNA library. Restriction sites for Pst1 and Kpn1 were added to facilitate directional subcloning into Bluebac 4.5 baculovrius vector (Invitrogen).

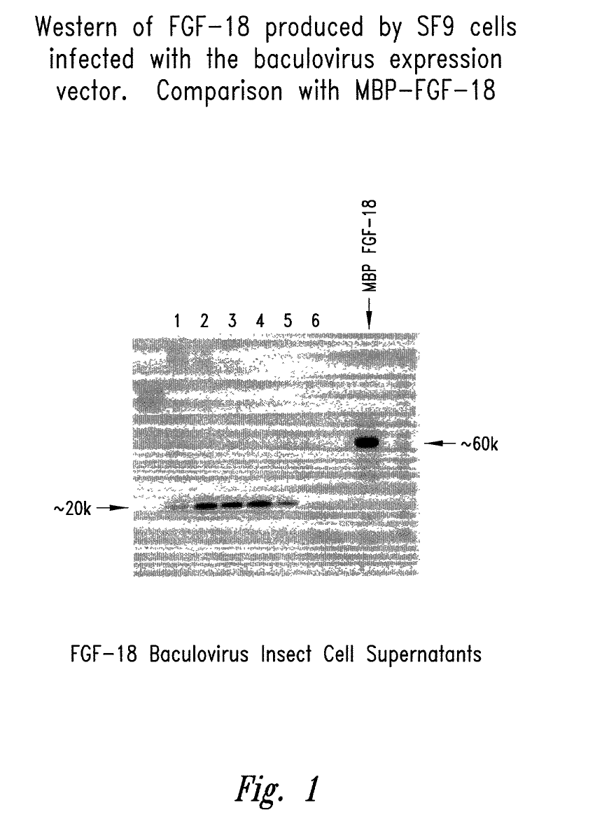

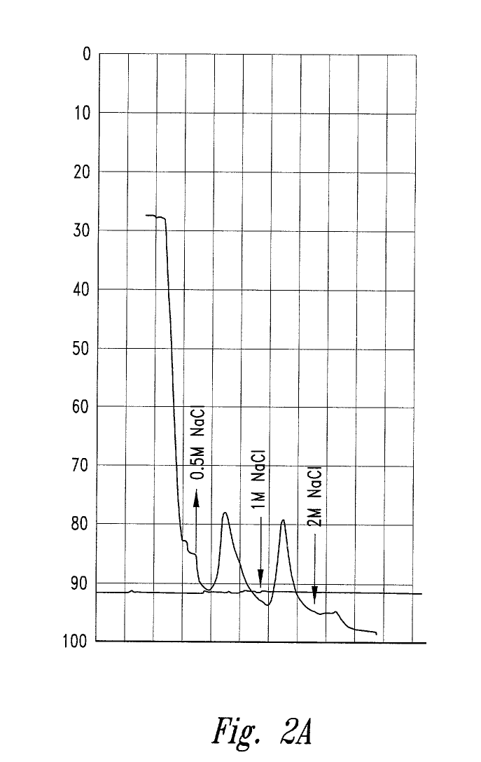

Supernatants from insect cells were generated for purification and bioassay by infecting SF9 cells at approximately 1-2×10⁶/ml with recombinant baculovirus derived from these plasmids. A multiplicity of infection (moi) of 2-5 was used for the 48 hour infections. SF9 cells were removed from the supernatant by centrifugation and the supernatants further clarified by 0.2 μM sterile filtration. In order to determine high producers, SF9 cells infected with different clones of baculovirus supernatants were analyzed by Western blot (FIG. 1). A peptide antibody directed against the C terminal region of FGF 98 (AA177 to 195) was used. The yield in insect cells was 4 to 8 mg protein per liter of CM.

### EXAMPLE 2

### Isolation of FGF 98

Recombinant FGF 98 from the conditioned medium of SF-9 cells was purified by Heparin Sepbarose (HS) affinity chromatography, followed by Mono S cation exchange chromatography. The recombinant FGF 98 bound to the HS was eluted using a step-wise salt gradient. Next, the recombinant FGF 98 eluted from the HS column was bound to the Mono S cation exchange column and was eluted using a nonlinear salt gradient.

Further details of the procedure utilized are described below.

A. Heparin Sepharose® Affinity Chromatography

The conditioned medium from Example 1 (30 ml) was loaded through a 5 ml bed of HS resin. The column was equilibrated in a buffer containing 150 mM NaCl and 10 mM Tris-HCl at pH 7.3. Once the conditioned medium was loaded, the column was washed extensively with the equilibration buffer until the absorbency at 280 nm returned to baseline. The protein was eluted from the HS column with an increasing step-wise NaCl gradient. The NaCl concentrations were 0.5M , 1M, and 2M NaCl, in 10 mM Tris-HCl at pH 7.3. The flow rate of the column during elution was approximately 30 ml/hr and 0.5-ml size fractions were collected.

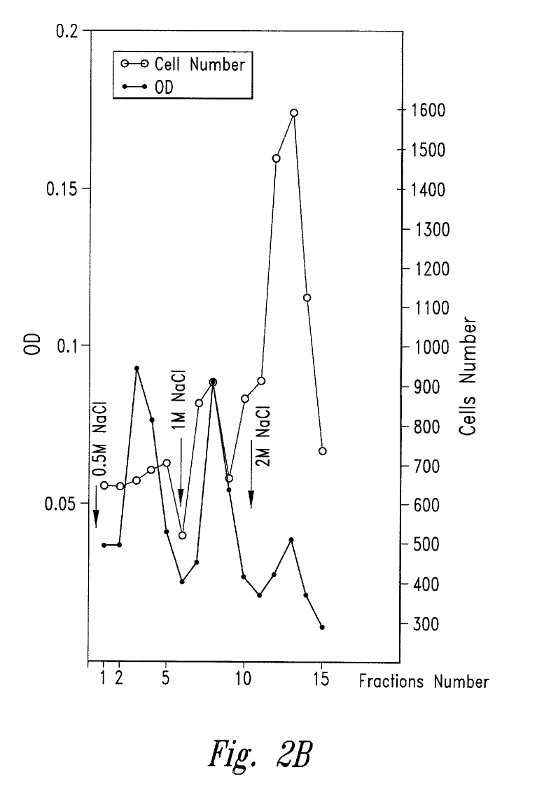

The fractions were tested for FGF 98 bioactivity utilizing granulosa cells. The assay is described in Section 4 below. The fractions with the highest bioactivity were eluted with 2M NaCl (FIG. 2).

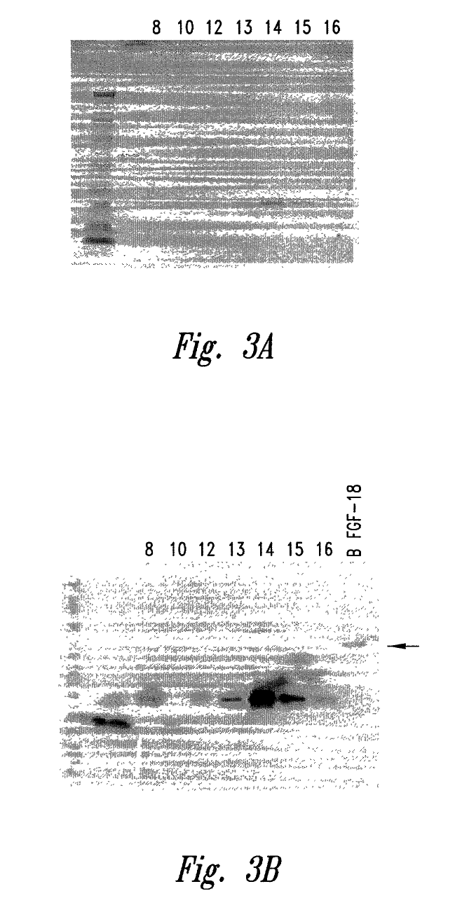

When analyzed by SDS-PAGE followed by Coomassie blue staining the peak 2M NaCl fractions gave a single band with a MW of 28 kDa (FIG. 3A). When analyzed by Western using an anti-FGF 98 peptide antibody the 2M NaCl fractions were immunoactive with a single band with a MW of 28 kDa (FIG. 3B). No immunoreactive bands could be observed with the 1M NaCl fractions.

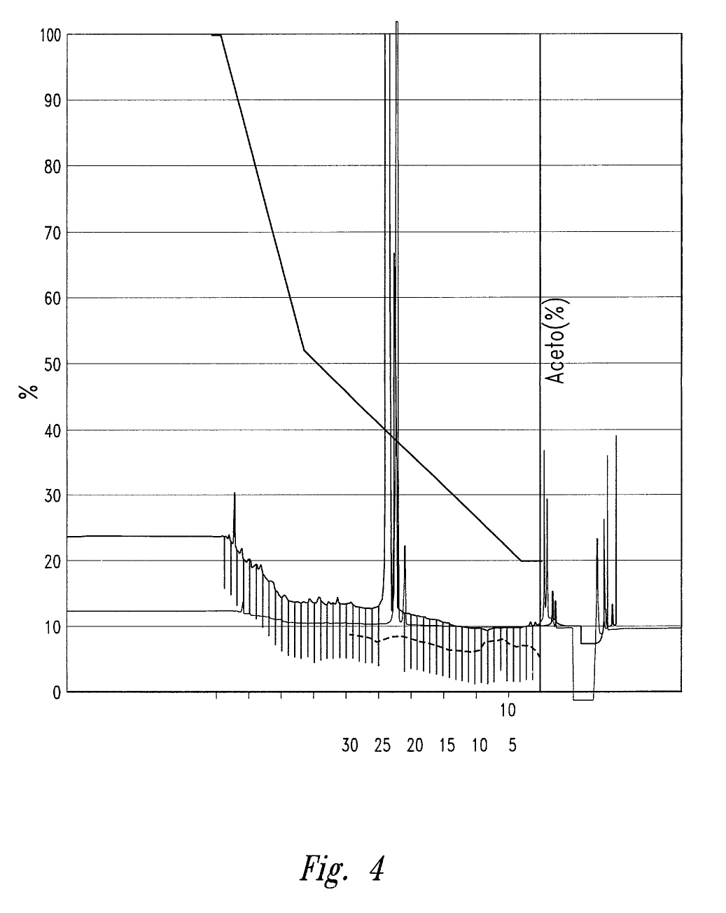

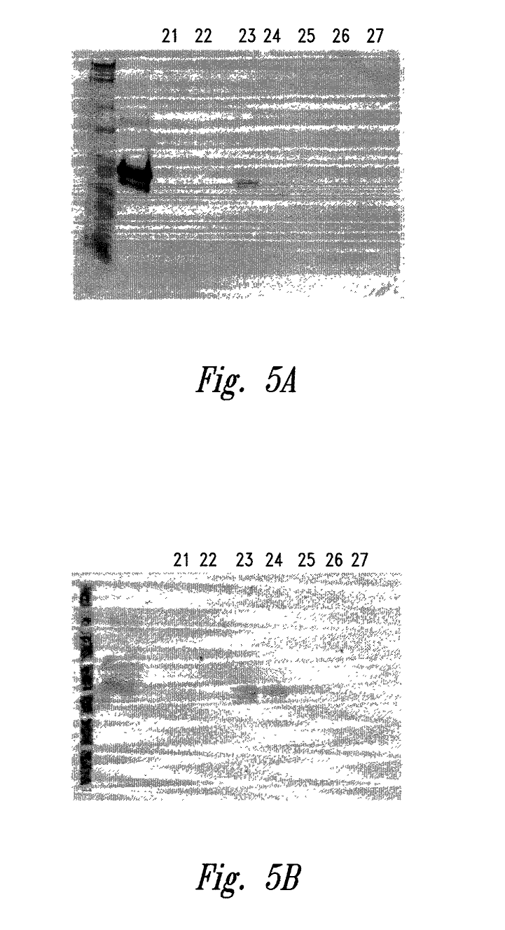

The homogeneity of the pooled 2M NaCl fractions was also analyzed by RP-HPLC. A single major protein peak eluting at 40% acetonitrile was observed. Closely associated with it was a minor protein peak that accounted for less than 5% of the protein mass (FIG. 4). When the HPLC fractions were analyzed for bioactivity using granuloma cells, both protein peaks were active. When analyzed by SDS-PAGE followed by Coomassie blue staining, both fractions gave a single band with an apparent MW of 28k (FIG. 5A). When this was followed by Western analysis both bands were recognized by a peptide antibody specific for FGF 98 (FIG. 5B).

However, when the bioactivity of the pooled 2M NaCl fractions was analyzed on vascular endothelial cells, cytotoxicity was observed, indicating the presence of endotoxin-like substances. Those were eliminated using Mono S cation exchange chromatography as described below.

B. Large Scale Isolation of FGF 98

A 3-liter batch of SF-9 cell-conditioned medium with pH adjusted to 7.3 and NaCl concentration raised to 0.5M was then allowed to run overnight at 4° C. through a 30 ml bed of HS resin. The column was equilibrated in a buffer containing 0.5M NaCl and 10 mM Tris-HCl pH 7.3. Following loading, the column was washed with the same buffer. The bound proteins were then eluted with 1M and 2M NaCl in 10 mM Tris pH 7.3. The flow rate of the column during the elution was 90 ml/hr and 3 ml size reactions were collected (FIGS. 6A, B).

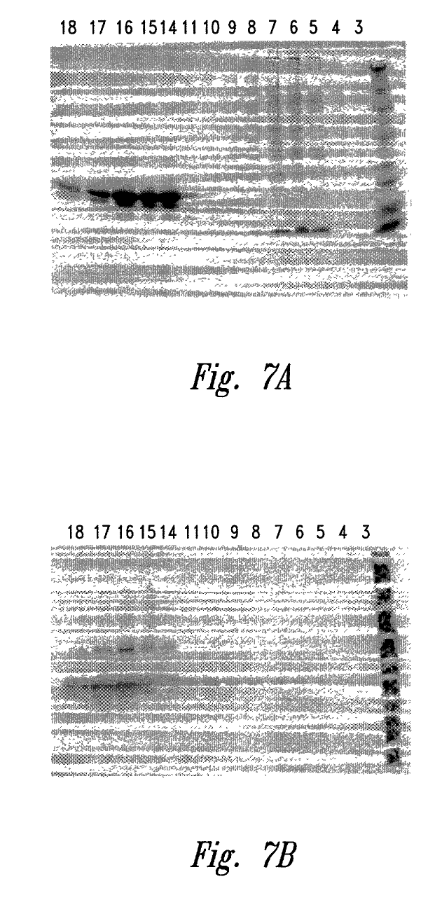

When the collected fractions were tested for bioactivity using granulosa cells, all of the bioactivity was present in the 2M NaCl fractions. When the 1M and 2M NaCl fractions were analyzed by SDS-PAGE, the 1M NaCl fraction showed multiple bands with MW ranging from 200 kDa to 8 kDa. In contrast, the 2M NaCl fractions show a major single band with a MW of 28 kDa (FIG. 7A). When analyzed by Western using an FGF 98 peptide antibody, the 1M NaCl contained a very weak immunoreactive band with a MW of 28 kDa. In contrast the 2M NaCl showed a very strong immunoreactive band at 28 kDa together with a weak band at 56 kDa (FIG. 7B).

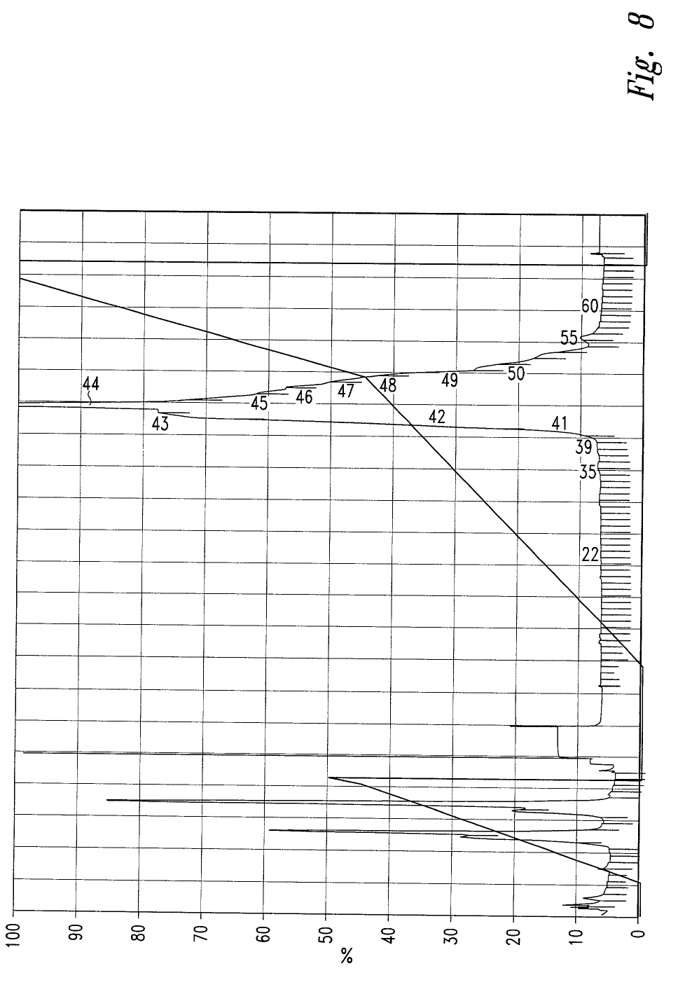

When the 2M NaCl fractions were tested on vascular endothelial cells, all fractions were toxic, indicating the presence of endotoxin-like material. Those were eliminated using Mono S cation exchange chromatography (FIG. 8).

C. Mono S Cation Exchange Chromatography

The pooled 2M NaCl fractions eluted from the HS column were diluted ten-fold with 10 mM Tris-HCl at pH 7.3 to a final salt concentration of 0.2M NaCl. The material was then loaded with a Super loop onto a Mono S column linked to a FPLC system (Pharmacia, Piscataway, N.J.). The Mono S cation exchange column was equilibrated with 10 mM Tris-HCl at pH 7.2. When the pooled fractions were loaded, the column was washed extensively at a flow rate of 1 ml/min. with the equilibration buffer until the absorbency returned to baseline. Then, protein was eluted from the column with a nonlinear NaCl gradient, 0.2M to 2M NaCl in 10 mM Tris-HCl at pH 7.3 at a flow rate of 1 ml/min and 1 ml fractions were collected.

One major protein peak of activity was observed that eluted at about 0.8M NaCl (FIG. 8). Fractions across the protein peak were assayed for bioactivity and subjected to SDS-PAGE analysis. The results of the gel analysis (not illustrated) showed that protein from NaCl fractions exhibited an apparent molecular weight of 28 kD.

Western analysis of the Mono S 0.8M pooled fraction with peptide antibodies directed against AA176 to AA190 of FGF 98 revealed a single band with an apparent MW of 28 kD.

The homogeneity of the pooled Mono S fractions was also analyzed by RP-HPLC using a C4 column equilibrated in 0.1% TFA, 20% acetonitrile, using an acetonitrile gradient of 20 to 100%. A profile similar to that shown in FIG. 4 was obtained (data not shown).

### EXAMPLE 3

### FGF 98 Bioactivity Effect on Granulosa Cells

Stock cultures of granulosa cells were grown and maintained in Dulbecco's modified Eagle medium supplemented with 10% bovine serum. 0 25 μl/ml fungizone, and 10 ng/ml FGF. The cells were incubated at 37° C. in a 10% CO₂ atmosphere with 99% humidity. For the bioactivity assay, the cells were seeded in 12-well plates at a density of 5×10³ cells per well in 1 ml of medium as described above for the stock cultures, and as described in Gospodarowicz et al. Meth. Enzymol. 147B:106-119 (D. Barnes et al., ed.) Academic Press, Orlando, Fla. (1987).

Ten microliter aliquots of the desired column fractions were diluted into 1 ml of 0.2% (w/v) gelatin in phosphate buffered saline (PBS). Ten microliters of this dilution were added to granulosa cells seeded in 12-well cluster plates containing 22 mm wells, at 5×10³ cells per well, and a 10 μl aliquot of either the diluted column fractions or medium containing 10 ng bFGF were added to the cells every other day.

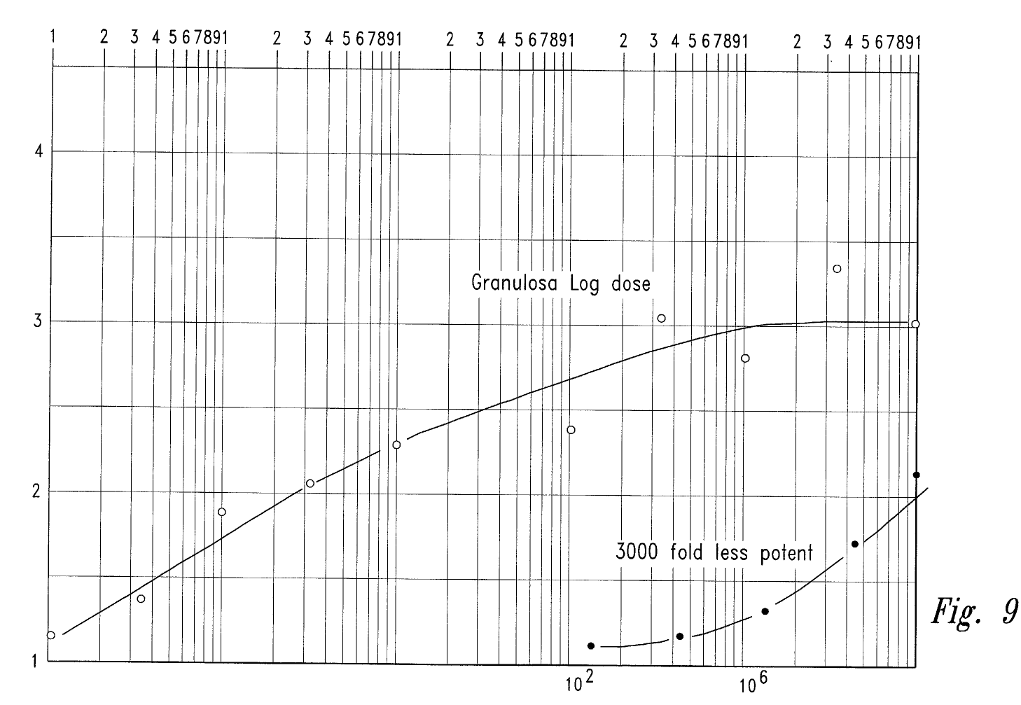

After five days in culture, the cells were trypsinized and the final cell density was determined using a Coulter® counter. (Coulter Electronics, Hialeah, Fla., USA). The cells were released from the plates by replacing the culture medium with a solution containing 0.9% NaCl, 0.01M sodium phosphate (pH 7.4), 0.05% trypsin, and 0.02% EDTA (STV). The cells were incubated in this solution for 5-10 minutes at 37° C., and then the stock culture medium was added to the cells. The cells were then counted using a Coulter® counter. Results shown in FIG. 9 demonstrates the effect of different dilutions of FGF 98 on granulosa cells as compared to basic FGF. The figure legend for FIG. 9 is as follows: O=basic FGF, ●=FGF 98.

The final cell density was graphed as a function of protein concentration. The protein concentration is graphed on a log scale. The protein concentration was determined by Bradford assay according to instructions accompanying the protein assay kit from BIORAD (Richmond, Calif., USA).

The ED₅₀ was calculated by (a), dividing in half the difference between the lowest and highest cell density value of the curve; and (b), determining from the graph what protein concentration corresponds to that cell density number obtained in (a). According to FIG. 9 the ED₅₀ for bFGF was found to be approximately 40 pg/ml, that for FGF 98 was about 100 ng/ml. The results of this assay demonstrate that FGF 98 was about 2500-fold less active than bFGF.

### EXAMPLE4

### Absence of FGF 98 Bioactivity on ABAE or ACE Cells

FGF 98 can be characterized by its lack of activity on vascular endothelial cells derived from large vessels (“ABAE”) or capillary cells (“ACE”) as compared with basic FGF (“bFGF”). Stock cultures of ABAE and ACE cells were grown and maintained in Dulbecco's modified Eagle medium supplemented with 10% bovine serum, 0.25 μl/ml fungizone, and 2 ng/ml bFGF. The cells were incubated at 37° C. with a 10% CO₂ concentration and 99% humidity.

In the mitogenic assay, either 10⁴ ABAE or 5×10³ ACE cells were plated per well in 12-well plates in stock culture medium, as described in Gospodarowicz et al., Proc. Natl. Acad. USA 73:4120-4124 (1976), Gospodarowicz et al., J. Cell. Physiol. 127:121-136 (1976); and Gospodarowicz et al., Proc. Natl. Acad. USA 86:7311-7315 (1989). Ten microliter aliquots of the fractions to be tested were diluted into 1 ml of 0.2% (w/v) gelatin in phosphate buffered saline. Ten microliters of this dilution were added to cells seeded in 12-well cluster plates. A 10 μl aliquot of either the diluted purified FGF 98 medium-containing fractions or 2 ng bFGF was added every other day.

After five days in culture, the cells were trypsinized and the final cell density was determined using a Coulter® counter. The cells were released from the plates by replacing the medium with a solution containing 0.9% NaCl, 0.01% M sodium phosphate (pH 7.4), 0.05% trypsin, and 0.02% EDTA (STV). The cells were incubated in this solution for 5-10 minutes at 37° C., and then the stock culture medium was added to the cells. The cells then were counted using a Coulter® counter (Coulter Electronics, Hialeah, Fla., USA).

In the absence of heparin, results demonstrated the lack of activity of FGF 98 on ABAE and ACE cells in contrast to bFGF. In the presence of heparin (2 μg/ml), FGF 98 was bioactive on ABAE as well as ACE cells.

### EXAMPLE 5

### FGF 98 Expression Patterns in Embryogenesis

To determine the expression pattern of FGF 98 during embryogenesis, wholemount in situ hybridizations were performed on mouse embryos ranging in stage from embryonic day (E) 7.5 to E.11.5 and on limbs from E12.5 to E14 5. For wholemount RNA in situ hybridizations, mouse embryos (CD-1) were isolated, fixed, and processed following the protocol by Nieto, M A., Patel, K., Wilkinson, D. G. 1995. In situ hybridization analysis of chick embryos in whole mount and tissue section. In: Methods in Avian Embryology (ed. M. Bronner-Fraser), San Diego, Academic Press. Antisense riboprobe for FGF 98 was synthesized using the cDNA corresponding to EST accession number AA656693 as a template, linearized with XbaI and transcribed with T7 RNA polymerase. This probe encompasses C-terminal coding sequence and 3′ untranslated region. The fgf-8 probe was prepared as described by Crossley et al., Development 121:439-451 (1995). Embryos processed for wholemount in situ hybridization were equilibrated in 30% sucrose, embedded in OCT or 15% sucrose, 7.5% gelatin and cryosectioned at 12 or 30 μm, respectively.

At E7.5, no FGF 98 transcripts were detectable. At E8.5, FGF 98 transcripts were present in the paraxial mesoderm and in the newly formed somites. At all subsequent stages analyzed, FGF 98 continued to be expressed in the rostral part of the paraxial mesoderm and throughout the newly formed somites. As the more rostral somites mature, FGF 98 transcripts first become restricted to the caudal part of the somite, and then to the caudal part of the myotome.

In the developing limbs, FGF 98 expression was initiated at E10.5 in a dorsal and a ventral domain in the mesenchyme. At E11, two separate domains of expression were observed on the ventral side of the limb. From E11.5 to at least E14.5, FGF 98 expression appeared to outline the differentiating skeletal elements, first the humerus, radius and ulna, followed by the digits. Transverse sections through E13.5 digits demonstrated that FGF 98 expression was present in tissues surrounding the differentiating cartilage elements. Furthermore, FGF was expressed in the interdigital regions at E12.5.

In the developing nervous system, FGF 98 transcripts were detected from E9.0 through at least E11.5. From E9.0 to E9.5, FGF 98 was transiently expressed in the caudal part of the neural plate which has not yet closed. In the differentiating neural tube, FGF 98 expression was initiated at E 9.5, in a dorsal and a ventral domain in the mantle layer. Furthermore, FGF 98 expression was observed in the dorsal root ganglia and in a subset of cranial ganglia (v, vii-viii, ix-x).

During the early development of the brain, the expression pattern of FGF 98 resembled that of fgf-8. At E8.5, FGF 98 expression was restricted to a narrow stripe in the head similar to a domain observed for fgf-8 transcripts (Crossley et al., Development 121:439-451 (1995)). At subsequent stages, this FGF 98 expression domain marked the border between the midbrain and the hindbrain. However, the expression domain of FGF 98 in this region appeared to be wider than that of fgf-8. In the commissural plate of the telencephalon, FGF 98 expression was initiated at E9.0, approximately 0.5 days later than that of fgf-8. In the diencephalon, FGF 98 transcripts were first detected at E9.5 in both a dorsal and a ventral domain, and by E10 5, dorsal expression extended from the diencephalon rostrally into the telencephalon. In the optic stalks, FGF 98 expression was restricted ventrally, similar to fgf-8.

Finally, FGF 98 mRNA was detected in the pharyngeal region beginning at E9.5 in a restricted region of the second and the third branchial arch.

### EXAMPLE 6

### Stem Cell Proliferation Assay

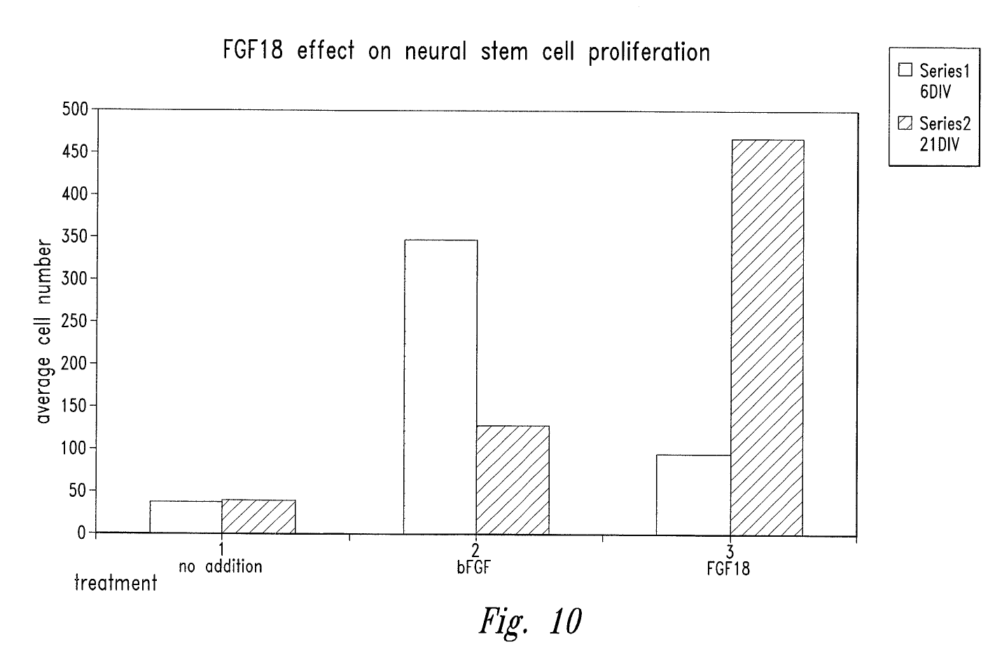

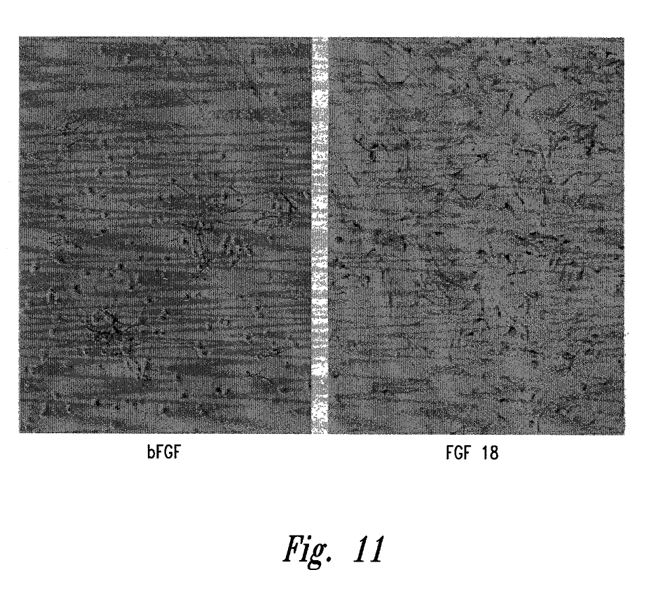

The neuron stem cell/progenitor assay involved the culturing of hippocampal cells from the embryonic rat brain for 6 days at 37° C. in medium containing FGF 98, basic FGF, or no added factor. Neuronal survival and proliferation were assessed visually under a microscope on day 6. Photomicrographs were made and the cell number in each condition counted (the data are shown in FIG. 10; series 1, 6 days in vitro DIV). The cultures were then washed and fresh media added without added FGF 98 or bFGF. Following 15 days in media without added factors cells were visually inspected, photomicrographs made and cells counted (FIG. 11, series 2, 21 DIV). FGF 98 increases the number of neural cells at both 6DIV and 21DIV. FGF 98 stimulates the proliferation of neural progenitors under these conditions. The effects of FGF 98 are significantly different than the effect of bFGF where there is proliferation at 6 DIV but many of these cells die following withdraw of the growth factor. Few cells die in the FGF 98 treatment conditions.

Based on these and other results, FGF 98 can be used for the ex vivo expansion of precursor cells on the nervous system. Transplant or engraftment of cells can be used as a therapy for diseases in which specific populations of neurons are lost due to degeneration such as, for example, Parkinson's disease and Alzheimer's disease.

### EXAMPLE 7

### Preparation of Primary Hippocampal Cell Cultures

Hippocampi were dissected from E18-19 rats. The tissue was collected in Hibernate™ media (Gibco BRL) and minced into approximately 1 mm pieces. Tissue was dissociated using the Papain Dissociation System (Worthington Biochemical Corporation). Following isolation the cells were resuspended in serum-free media composed of Neurobasal™ (Gibco BRL), 2% B27 supplement (GibcoBRL), L-glutamate and antibiotics. At the time of plating the growth factors were added bFGF at 10 ng/ml or FGF 98 at 200 ng/ml. Cells were plated in 35 mm tissue culture dishes coated with poly-L-lysine at a concentration of 7.5×10⁴ cells per dish. Following 6 days at 37° C. in 5% CO₂ cells were rinsed and fed with fresh media; no growth factors were added. Fifteen days after the factors were removed (total 21 DIV) the cells were fixed for 30 min in 4% paraformaldehyde and photographed. Some cultures are stained with the monoclonal antibody to the nestin intermediate filament (Developmental Studies Hybridoma Bank). This antibody recognizes neural stem cells/precursors. (Johe, K. K., et al. (1996). Genes and Development 10:3129-3140.) FIG. 11 shows the expression of nestin in 15 DIV cultures treated with either bFGF or FGF 98 for the first 6 days. There are many more nestin positive cells in the FGF 98 treated cultures. See FIG. 11, Panel A, nestin expression in bFGF treated cultures, and FIG. 11, Panel B, nestin expression in FGF 98 treated cultures.

### EXAMPLE 8

### FGF 98 Expression

A survey of FGF 98 expression was performed in mouse embryonic and adult tissues (>3 months) using in situ hybridization. FGF 98 is expressed in the E14 mouse nervous system; areas of expression include the motor neurons of the brainstem and spinal cord, the dorsal root and cranial ganglia of the peripheral nervous system, the ventral tegmentum, developing forebrain, the region of the isthmus and developing cerebellum. In the adult brain FGF 98 is expressed in both the brain and spinal cord including the motor neurons, the substantia nigra, the deep cerebellar nuclei, the red nucleus and other areas. There are also several non-neuronal tissues in which FGF 98 is expressed including lung, heart, developing bone and chondrocytes.

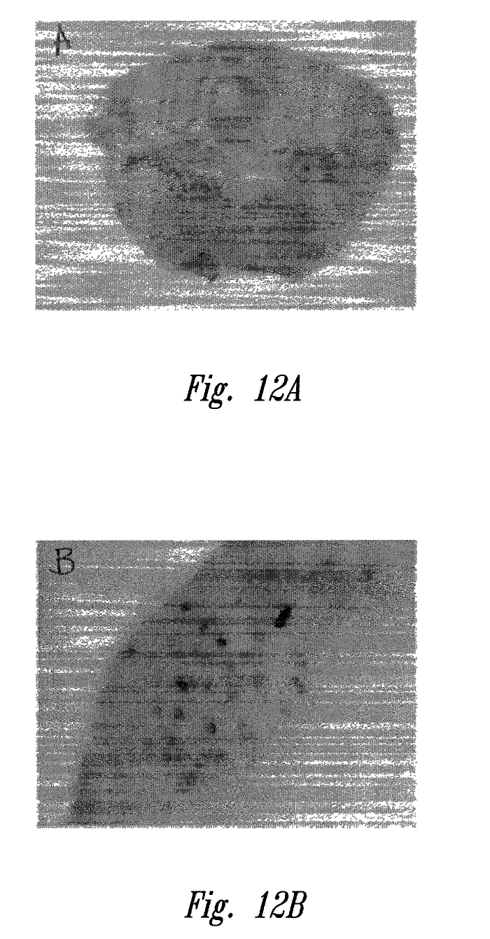

FIG. 12, Panel A, shows in situ hybridization of the adult brain stem and cerebellum. FGF 98 mRNA expression in deep cerebellar nuclei, and motor neurons of the facial and cochlear nuclei is visible. FIG. 12, Panel B shows a high magnification of FGF 98 expression in motor neurons of the facial nucleus.

### EXAMPLE 9

### Preparation of Antisera to FGF 98 by Immunization of Rabbits with an FGF 98 Peptide

The peptide sequence corresponding to amino acids 176-190 of the human FGF 98 protein was synthesized and coupled to keyhole limpet hemocyanin (KLH) as described (Harlow and Land, Antibodies: A Laboratory Manual, 1988. Cold Spring Harbor Laboratory, New York, N.Y.). The KLH-coupled peptide was submitted to EL Labs and each of three rabbits were immunized. (EL Labs, Soquel, Calif.).

Peptide sequence: H-KRYPKGQPELQKPFK-OH (KLH) peptide, synthesized by Research Genetics, Huntsville, Ala.

Deposit Information

The following materials were deposited with the American Type Culture Collection:

| Name | Deposit Date | Accession No. |
|---|---|---|
| Escherichia coli 5 a FGF98 | February 26, | 98662 |
| | 1998 | |
| Escherichia coli DH5-α FGF8h#4 | February 18, | 98677 |
| | 1998 | |

The above materials have been deposited with the American Type Culture Collection, 10801 University Blvd., Manassas, Va. 20110-2209, telephone 703-365-2700, under the accession numbers indicated. This deposit will be maintained under the terms of the Budapest Treaty on the International Recognition of the Deposit of Microorganisms for purposes of Patent Procedure. The deposits will be maintained for a period of 30 years following issuance of this patent, or for the enforceable life of the patent, whichever is greater. Upon issuance of the patent, the deposits will be available to the public from the ATCC without restriction.

These deposits are provided merely as convenience to those of skill in the art, and are not an admission that a deposit is required under 35 U.S.C. §112. The sequence of the polynucleotides contained within the deposited materials, as well as the amino acid sequence of the polypeptides encoded thereby, are incorporated herein by reference and are controlling in the event of any conflict with the written description of sequences herein. A license may be required to make, use, or sell the deposited materials, and no such license is granted hereby.

All patents, published patent applications and publications cited herein are incorporated by reference as if set forth fully herein.

Although certain preferred embodiments have been described herein, it is not intended that such embodiments be construed as limitations on the scope of the invention except as set forth in the following claims.

### POLYNUCLEOTIDE AND POLYPEPTIDE SEQUENCES

SEQ ID NO:1 is the nucleotide sequence.

The reading frame starts at nucleotide 610

CTATAGAAGGTACGCCTGCAGGTACCGGTCCGGAATTCCCGGGTCGACCCACGCGTCCGCCCACGCGTCCGCGGACGCGTGGGGACGCG
GAGGAGGAGACATGAGCCGGCGGGCGCCCAGACGGAGCGGCCGTGACGCTTTCGCGCTGCAGCCGCGCGCCCCGACCCCGGAGCGCTGA
CCCCTGGCCCCACGCAGCTCCGCGCCCGGGCCGGAGAGCGCAACTCGGCTTCCAGACCCGCCGCGCATGCTGTCCCCGGACTGAGCCGG
GCAGCCAGCCTCCCACGGACGCCCGGACGGCCGGCCGGCCAGCAGTGAGCGAGCTTCCCCGCACCGGCCAGGCGCCTCCTGCACAACGG
CTGCCGCCCCGCAGCCCCTGCGCCAGCCCGGAGGGCGCAGCGCTCGGGAGGAGCCGCGCGGGGCGCTGATGCCGCAGGGCGCGCCGCGG
AGCGCCCCGGAGCAGCAGAGTCTGCAGCAGCAGCAGCCGGCGAGGAGGGAGCAGCAGCAGCGGCGGCGGCGGCGGCGGCGGCGGCGGAG
GCGCCCGGTCCCGGCCGCGCGGAGCGGACATGTGCAGGCTGGGCTAGGAGCCGCCGCCTCCCTCCCGCCCAGCGATGTATTCAGCGCCC
TCCGCCTGCACTTGCCTGTGTTTACACTTCCTGCTGCTGTGCTTCCAGGTACAGGTCCTGGTTGCCGAGGAGAACGTGGACTTCCGCAT
CCACGTGGAGAACCAGACGCGGGCTCGGGACGATGTGAGCCGTAAGCAGCTGCGGCTGTACCAGCTCTACAGCCGGACCAGTGGGAAAC
ACATCCAGGTCCTGGGCCGCAGGATCAGTGCCCGCGGCGAGGATGGGGACAAGTATGCCCAGCTCCTAGTGGAGACAGACACCTTCGGT
AGTCAAGTCCGGATCAAGGGCAAGGAGACGGAATTCTACCTGTGCATGAACCGCAAAGGCAAGCTCGTGGGGAAGCCCGATGGCACCAG
CAAGGAGTGTGTGTTCATCGAGAAGGTTCTGGAGAACAACTACACGGCCCTGATGTCGGCTAAGTACTCCGGCTGGTACGTGGGCTTCA
CCAAGAAGGGGCGGCCGCTCTAGAGGATCCCTCGAGGGGCCCAAGCTTACGCGTGCATGC
SEQ ID NO:2 is the amino acid sequence
MYSAPSACTCLCLHFLLLCFQVQVLVAEENVDFRIHVENQTRARDDVSRKQLRLYQLYSRTSGKHIQVLGRRISARGEDGDKYAQLLVE
TDTFGSQVRIKGKETEFYLCMNRKGKLVGKPDGTSKECVFIEKVLENNYTALMSAKYSGWYVGFTKKGRPL
SEQ ID NO:3 is the primer sequence
GCGAGGATGGGGACAAGTAT
SEQ ID NO:4 is the FGF coding region with the native human 5′ and 3′ untranslated region
CCCACGCGTCCGCGGACGCGTGGGGACGCGGAGGAGGAGACATGAGCCGGCGGGCGCCCAGACGGAGCGG
CCGTGACGCTTTCGCGCTGCAGCCGCGCGCCCCGACCCCGGAGCGCTGACCCCTGGCCCCACGCAGCTCC
GCGCCCGGGCCGGAGAGCGCAACTCGGCTTCCAGACCCGCCGCGCATGCTGTCCCCGGACTGAGCCGGGC
AGCCAGCCTCCCACGGACGCCCGGACGGCCGGCCGGCCAGCAGTGAGCGAGCTTCCCCGCACCGGCCAGG
CGCCTCCTGCACAACGGCTGCCGCCCCGCAGCCCCTGCGCCAGCCCGGAGGGCGCAGCGCTCGGGAGGAG
CCGCGCGGGGCGCTGATGCCGCAGGGCGCGCCGCGGAGCGCCCCGGAGCAGCAGAGTCTGCAGCAGCAGC
AGCCGGCGAGGAGGGAGCAGCAGCAGCGGCGGCGGCGGCGGCGGCGGCGGCGGAGGCGCCCGGTCCCGGC
CGCGCGGAGCGGACATGTGCAGGCTGGGCTAGGAGCCGCCGCCTCCCTCCCGCCCAGCGATGTATTCAGC
GCCCTCCGCCTGCACTTGCCTGTGTTTACACTTCCTGCTGCTGTGCTTCCAGGTACAGGTGCTGGTTGCC
GAGGAGAACGTGGACTTCCGCATCCACGTGGAGAACCAGACGCGGGCTCGGGACGATGTGAGCCGTAAGC
AGCTGCGGCTGTACCAGCTCTACAGCCGGACCAGTGGGAAACACATCCAGGTCCTGGGCCGCAGGATCAG
TGCCCGCGGCGAGGATGGGGACAAGTATGCCCAGCTCCTAGTGGAGACAGACACCTTCGGTAGTCAAGTC
CGGATCAAGGGCAAGGAGACGGAATTCTACCTGTGCATGAACCGCAAAGGCAAGCTCGTGGGGAAGCCCG
ATGGCACCAGCAAGGAGTGTGTGTTCATCGAGAAGGTTCTGGAGAACAACTACACGGCCCTGATGTCGGC
TAAGTACTCCGGCTGGTACGTGGGCTTCACCAAGAAGGGGCGGCCGCGGAAGGGCCCCAAGACCCGGGAG
AACCAGCAGGACGTGCATTTCATGAAGCGCTACCCCAAGGGGCAGCCGGAGCTTCAGAAGCCCTTCAAGT
ACACGACGGTGACCAAGAGGTCCCGTCGGATCCGGCCCACACACCCTGCCTAGGGCACCCCGCCGCGGCC
CCTCAGGTCGCCCTGGCCACACTCACACTCCCAGAAAACTGCATCAGAGGAATATTTTTACATGAAAAAT
AAGGAAGAAGCTCTATTTTTGTACATTGTGTTTAAAAGAAGACAAAAACTGAACCAAAACTCTTGGGGGG
AGGGGTGATAAGGATTTTATTGTTGACTTGAAACCCCCGATGACAAAAGACTCACGCAAAGGGACTGTAG
TCAACCCACAGGTGCTTGTCTCTCTCTAGGAACAGACAACTCTAAACTCGTCCCCAGAGGAGGACTTGAA
TGAGGAAACCAACACTTTGAGAAGCCAAAGTCCTTTTTCCCAAAGGTTCTGAAAGGAAAAAAAAAAAAAC
AAAAAAAAAAAAAAAAAAAAAAAAAAAAAA
SEQ ID NO:5 is the fill length amino acid sequence of FGF
MYSAPSACTCLCLHFLLLCFQVQVLVAEENVDFRIHVENQTRARDDVSRKQLRLYQLYSRTSGKHIQVLG
RRISARGEDGDKYAQLLVETDTFGSQVRIKGKETEFYLCMNRKGKLVGKPDGTSKECVFIEKVLENNYTA
LMSAKYSGWYVGFTKKGRPRKGPKTRENQQDVHFMKRYPKGQPELQKPFKYTTVTKRSRRIRPTHPA

We claim:

1. An isolated polynucleotide encoding a fibroblast growth factor polypeptide selected from the group consisting of:
(a) a polynucleotide comprising the nucleotide sequence of SEQ ID NO:4;
(b) a polynucleotide that encodes a variant of the polypeptide encoded by (a);
(c) a polynucleotide encoding a protein expressed by a polynucleotide having the sequence of SEQ ID NO:4; and
(d) a polynucleotide molecule that encodes a polypeptide that is at least 65% identical to the amino acid sequence of SEQ ID NO:4.

2. The polynucleotide of claim 1 having the nucleotide sequence of SEQ ID NO:4.

3. An expression vector comprising a polynucleotide according to claim 1 operably linked to a transcriptional promoter and a transcriptional terminator.

4. A host cell comprising the vector of claim 3 wherein said host cell is a mammalian cell, a bacterial cell, a yeast cell, or an insect cell.

5. An isolated polypeptide selected from the group consisting of:
(a) a polypeptide encoded by SEQ ID NO:4;
(b) a variant of the protein (a) or (b); and
(c) a polypeptide that is at least 65% identical to the amino acid sequence of SEQ ID NO:5.

6. A method of producing an FGF 98 polypeptide, said method comprising:
(a) culturing a host cell according to claim 4, wherein said cell expresses an FGF 98 polypeptide encoded by said vector; and
(b) recovering the FGF 98 polypeptide.

7. The method of claim 6 wherein said cell is an insect cell.

8. A pharmaceutical composition comprising the FGF 98 polypeptide of claim 5, in combination with a pharmaceutically acceptable carrier.

9. An antibody that binds to the polypeptide of claim 5 wherein said antibody is selected from the group consisting of a polyclonal antibody, a monoclonal antibody, and a single chain antibody.

10. The antibody of claim 9 wherein said antibody is raised against an immunogen comprising the amino acid sequence KRYPKGOPELQKPFK (SEQ ID NO:6).

11. A method for providing trophic support for cells in a patient in need thereof, the method comprising administering to the patient a composition selected from the group consisting of a polynucleotide encoding an FGF 98 polypeptide comprising SEQ ID NO: 5, and a polypeptide according to claim 5.

12. The method of claim 11 wherein said polynucleotide is administered by implanting cells which express said polynucleotide into the patient, wherein said cells express FGF 98 polypeptide in the patient.

13. The method of claim 12 wherein the implanted cells are encapsulated in a semipermeable membrane.

14. The method of claim 11 wherein the patient suffers from a condition selected from the group consisting of peripheral neuropathy, amyotrophic lateral sclerosis, Alzheimer's disease, Parkinson's disease, Huntington's disease, ischemic stroke, brain injury, acute spinal cord injury, nervous system tumors, multiple sclerosis, infection, dementia, epilepsy, and peripheral nerve injury.

15. The method of claim 14 wherein the condition is Parkinson's disease.

16. The method of claim 14 wherein the condition is Alzheimer's disease.

17. The method of claim 14 wherein the condition is stroke.

18. The method of claim 14 wherein the condition is brain injury.

19. The method of claim 14 wherein the condition is spinal cord injury.

20. A kit for detecting the presence of mRNA encoding FGF 98 in a sample from a patient, said kit comprising a polynucleotide having at least 20 contiguous nucleotides of the polynucleotide of claim 1, packaged in a container.

21. The kit according to claim 20 wherein the polynucleotide encodes SEQ ID NO:2 or SEQ ID NO:5.

22. A kit for detecting the presence of FGF 98 polypeptide in a sample from a patient, said kit comprising an antibody according to claim 9, packaged in a container.


SEQUENCE LISTING (6 sequences)

SEQ ID NO: 1 - DNA, 1128 bp, Homo Sapien
ctatagaaggtacgcctgcaggtaccggtccggaattcccgggtcgacccacgcgtccgcccacgcgtccgcggacgcgtggggacgcggaggaggagacatgagccggcgggcgcccagacggagcggccgtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacaacggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgctctagaggatccctcgaggggcccaagcttacgcgtgcatgc

SEQ ID NO: 2 - protein, 160 aa, Homo sapien
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPL

SEQ ID NO: 3 - DNA, 20 bp, Artificial Sequence
  Primer sequence
gcgaggatggggacaagtat

SEQ ID NO: 4 - DNA, 1570 bp, Homo sapien
cccacgcgtccgcggacgcgtggggacgcggaggaggagacatgagccggcgggcgcccagacggagcggccgtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacaacggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggcccctcaggtcgccctggccacactcacactcccagaaaactgcatcagaggaatatttttacatgaaaaataaggaagaagctctatttttgtacattgtgtttaaaagaagacaaaaactgaaccaaaactcttggggggaggggtgataaggattttattgttgacttgaaacccccgatgacaaaagactcacgcaaagggactgtagtcaacccacaggtgcttgtctctctctaggaacagacaactctaaactcgtccccagaggaggacttgaatgaggaaaccaacactttgagaagccaaagtcctttttcccaaaggttctgaaaggaaaaaaaaaaaaacaaaaaaaaaaaaaaaaaaaaaaaaaaaaaa

SEQ ID NO: 5 - protein, 207 aa, Homo sapien
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 6 - protein, 15 aa, Homo sapien
KRYPKGQPELQKPFK